(19) United States
(12) Patent Application Publication
(10) Pub. No.: US 2006/0234969 A1
(43) Pub. Date: Oct. 19, 2006

(54) Compositions and methods for inhibiting inflammation of vessel walls and formation of neointimal hyperplasia

(75) Inventor: Kensuke Egashira, Fukuoka-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AnGes MG, INC., Ibaraki-shi (JP)

(21) Appl. No.: 10/548,369

(22) PCT Filed: Mar. 5, 2004
(86) PCT No.: PCT/JP04/02930

§ 371 Date: Jun. 23, 2006

Related U.S. Application Data
(60) Provisional application No. 60/452,720, filed on Mar. 7, 2003.

(51) Int. Cl. A61K 48/00

(52) U.S. Cl. 514/44; 424/93.2

(57) ABSTRACT

Compositions and methods for inhibiting inflammation of vessel wall and/or formation of neointimal hyperplasia by gene therapy using a soluble Flt-1 (sFlt-1) gene, are provided. VEGF has an essential role in the development of neointimal hyperplasia by causing inflammation. sFlt-1 gene transfer to the site of vascular injury blocks Flt-1-mediated VEGF signal transduction, thereby inhibiting early inflammation as well as late neointimal hyperplasia. The present invention is useful for inhibiting or treating inflammation of vessel wall and/or formation of neointimal hyperplasia in a patient with risk of post coronary intervention restenosis, atherosclerosis, arteriosclerosis, or edema.

### TECHNICAL FIELD

The present invention relates to gene therapy, and more specifically to compositions and methods for inhibiting or treating formation of neointimal hyperplasia using a gene encoding a soluble fragment of fms-like tyrosine kinase-1 (Flt-1).

### BACKGROUND ART

Neointimal hyperplasia (NIH) is a major cause of restenosis after coronary intervention (Libby P, Ganz P. Restenosis revisited—new targets, new therapies. N Engl J. Med. 1997;337:418-9; and Topol E J, Serruys P W. Frontiers in interventional cardiology. Circulation. 1998;98:1802-20). Vascular endothelial growth factor (VEGF) and its receptors (VEGFR-1: fms-like tyrosine kinase 1 receptor (Flt-1), VEGFR-2: endothelial type 2 receptor (Flk-1)) are upregulated in vascular inflammatory and proliferative disorders such as atherosclerosis and restenosis (Shibata M, Suzuki H, Nakatani M, Koba S, Geshi E, Katagiri T, Takeyama Y. The involvement of vascular endothelial growth factor and flt-1 in the process of neointimal proliferation in pig coronary arteries following stent implantation. Histochem Cell Biol. 2001;116:471-81; Ruef J, Hu Z Y, Ym L Y, Wu Y, Hanson SR, Kelly AB, Harker L A, Rao G N, Runge M S, Patterson C. Induction of vascular endothelial growth factor in balloon-injured baboon arteries. A novel role for reactive oxygen species in atherosclerosis. Circ Res. 1997;81:24-33; Chen Y X, Nakashima Y, Tanaka K, Shiraishi S, Nakagawa K, Sueishi K. Immunohistochemical expression of vascular endothelial growth factor/vascular permeability factor in atherosclerotic intimas of human coronary arteries. Arterioscler Thromb Vasc Biol. 1999;19:131-9; and Inoue M, Itoh H, Ueda M, Naruko T, Kojima A, Komatsu R, Doi K, Ogawa Y, Tamura N, Takaya K, Igaki T, Yamashita J, Chun T H, Masatsugu K, Becker A E, Nakao K. Vascular endothelial growth factor (VEGF) expression in human coronary atherosclerotic lesions: possible pathophysiological significance of VEGF in progression of atherosclerosis. Circulation. 1998;98:2108-16). VEGF is thought to protect the artery from such disorders by inducing endothelial regeneration and improving endothelial function mainly through the endothelial type 2 receptor Flk-1, and VEGF gene transfer or administration of its protein induces endothelial regeneration and attenuates NIH after endothelial injury (Baumgartner I, Isner J M. Somatic gene therapy in the cardiovascular system. Annu Rev Physiol. 2001;63:427-50). There is still considerable debate, however, over the role of VEGF in the development of NIH after injury (Isner J M. Still more debate over VEGF. Nat Med. 2001;7:639-41; and Ware J A. Too many vessels? Not enough? The wrong kind? The VEGF debate continues. Nat Med. 2001;7:403-4). Emerging evidence suggests that VEGF causes or promotes the development of atherosclerosis or NIH after injury. VEGF induces migration and activation of monocytes (Barleon B, Sozzani S, Zhou D, Weich H A, Mantovani A, Marme D. Migration of human monocytes in response to vascular endothelial growth factor (VEGF) is mediated via the VEGF receptor Flt-1. Blood. 1996;87:3336-43), adhesion molecules (Kim I, Moon S O, Kim S H, Kim H J, Koh Y S, Koh G Y. Vascular endothelial growth factor expression of intercellular adhesion molecule 1 (ICAM-1), vascular cell adhesion molecule 1 (VCAM-1), and E- selectin through nuclear factor-kappa B activation in endothelial cells. J Biol. Chem. 2001;276:7614-20), or monocyte chemoattractant protein-1 (MCP-1) (Marumo T, Schini-Kerth V B, Busse R. Vascular endothelial growth factor activates nuclear factor-kappaB and induces monocyte chemoattractant protein-1 in bovine retinal endothelial cells. Diabetes. 1999;48:1131-7), through its receptor Flt-1. Moreover, administration of VEGF protein to hypercholesterolemic animals enhances atherogenesis by inducing monocyte infiltration and activation (Celletti F L, Waugh J M, Amabile P C, Brendolan A, Hilfiker P R, Dake M D. Vascular endothelial growth factor enhances atherosclerotic plaque progression. Nat Med. 2001;7:425-9). In addition, VEGF might promote migration of vascular smooth muscle cells though Flt-1 (Grosskreutz C L, Anand-Apte B, Duplaa C, Quinn T P, Terman B I, Zetter B, D'Amore P A. Vascular endothelial growth factor-induced migration of vascular smooth muscle cells in vitro. Microvasc Res. 1999;58:128-36; and Ishida A, Murray J, Saito Y, Kanthou C, Benzakour O, Shibuya M, Wijelath E S. Expression of vascular endothelial growth factor receptors in smooth muscle cells. J Cell Physiol. 2001;188:359-68).

Various functions of Flt-1 have also been reported. Flt-1 in monocytes mediates chemotaxis (Barleon B et al., supra) and Flt-1 in smooth muscle cells mediates migration (Ishida A et al. supra and Wang H, Keiser J A Vascular endothelial growth factor upregulates the expression of matrix metalloproteinases in vascular smooth muscle cells: role of flt-1. Circ Res. 1998;83:832-40). Flt-1 acts as an important mediator of chemotaxis through VCAM-1, ICAM-1, and MCP-1 (Barleon B et al., supra, Kim I et al., supra, and Marumo T et al., supra). Luttun et al reported that treatment with anti-Flt-1 antibody attenuated the development of experimental tumor angiogenesis, arthritis, and atherosclerosis (Luttun A, Tjwa M, Moons L, Wu Y, Angelillo-Scherrer A, Liao F, Nagy J A, Hooper A, Priller J, De Klerck B, Compernolle V, Daci E, Bohlen P, Dewerchin M, Herbert J M, Fava R, Matthys P, Carmeliet G, Collen D, Dvorak H F, Hicklin D J, Carmeliet P. Revascularization of ischemic tissues by PlGF treatment, and inhibition of tumor angiogenesis, arthritis and atherosclerosis by anti-Flt1. Nat Med. 2002;8:831-40).

One reason for the inconsistent reports regarding the role of VEGF might be due to the fact that there are no selective VEGF inhibitors tested. The only known endogenous VEGF inhibitor is a soluble form of Flt-1 (sFlt-1), and this isoform is mainly expressed by vascular endothelial cells and can inhibit VEGF activity by directly sequestering VEGF and by functioning as a dominant negative inhibitor (Kendall R L, Wang G, Thomas K A. Identification of a natural soluble form of the vascular endothelial growth factor receptor, FLT-1, and its heterodimerization with KDR. Biochem Biophys Res Commun. 1996;226:324-8). It has also been shown that sFlt-1 has angiostatic properties by way of its antagonist activity against VEGF, probably because it binds VEGF but also because it binds and blocks the external domain of the membrane-bound Flt-1 (Kendall R L & Thomas K A. Inhibition of vascular endothelial cell growth factor activity by an endogenously encoded soluble receptor. Proc Natl Acad Sci USA. 1993 Nov. 15;90(22):10705-9, Goldman C K, Kendall R L, Cabrera G, Soroceanu L, Heike Y, Gillespie G Y, Siegal G P, Mao X, Bett A J, Huckle W R, Thomas K A, Curiel D T. Paracrine expression of a native soluble vascular endothelial growth factor receptor inhibits tumor growth, metastasis, and mortality rate. Proc Natl Acad Sci USA. 1998;95:8795-800, WO94/21679).

WO98/13071 discloses gene therapy methodology for inhibition of primary tumor growth and metastasis by gene transfer of a nucleotide sequence encoding a soluble form of a VEGF tyrosine kinase receptor to a mammalian host.

### DISCLOSURE OF THE INVENTION

An object of the present invention is to clarify the role of VEGF in the development of NIH and to provide compositions and methods for inhibiting inflammation of vessel walls and/or formation of NIH. The present inventor previously demonstrated that intramuscular transfection of the sFit-1 gene effectively and specifically blocks VEGF signaling, and thus quenches VEGF activity in vivo (Zhao Q, Egashira K, Inoue S, Usui M, Kitamoto S, Ni W, Ishibashi M, Hiasa Ki K, Ichiki T, Shibuya M, Takeshita A. Vascular endothelial growth factor is necessary in the development of arteriosclerosis by recruiting/activating monocytes in a rat model of long-term inhibition of nitric oxide synthesis. Circulation. 2002;105:1110-5, and Goldman C K et al. supra). Subsequently, the present inventor investigated the role of VEGF in the pathogenesis of NIH following cuff-induced periarterial injury in hypercholesterolemic mice. This cuff model was chosen because cuff placement in the presence of hypercholesterolemia offers the advantage of inducing reproducible site-controlled NIH and remodeling, and also the cuff-induced injury triggers vascular inflammation and induces neointimal lesions that are similar to the restenotic and atherosclerotic lesions observed in humans (Lardenoye J H, Delsing D J, de Vries M R, Deckers M M, Princen H M, Havekes L M, van Hinsbergh V W, van Bockel J H, Quax P H. Accelerated atherosclerosis by placement of a perivascular cuff and a cholesterol-rich diet in ApoE*3Leiden transgenic mice. Circ Res. 2000;87:248-53; and von der Thusen J H, van Berkel T J, Biessen E A. Induction of rapid atherogenesis by perivascular carotid collar placement in apolipoprotein E-deficient and low-density lipoprotein receptor-deficient mice. Circulation. 2001;103:1164-70).

The present inventor found that blockade of VEGF by sFlt-1 gene transfer reduced the early inflammatory and proliferative changes and thus attenuated the development of NIH. Perivascular inflammation has a major role in the pathogenesis of cuff-induced NIH (Egashira K, Zhao Q. Kataoka C, Ohtani K, Usui M, Charo I F, Nishida K, Inoue S, Katoh M, Ichiki T, Takeshita A. Importance of monocyte chemoattractant protein-1 pathway in neointimal hyperplasia after periarterial injury in mice and monkeys. Circ Res. 2002;90:1167-72, and Wu L, Iwai M, Nakagami H, Li Z, Chen R, Suzuki J, Akishita M, de Gasparo M, Horiuchi M. Roles of angiotensin II type 2 receptor stimulation associated with selective angiotensin II type 1 receptor blockade with valsartan in the improvement of inflammation-induced vascular injury. Circulation. 2001;104:2716-21). Accordingly, vascular inflammation and proliferation mediated through increased expression and VEGF activity are essential in the pathogenesis of NIH after cuff-induced perivascular injury.

VEGF is conventionally thought to be an endothelial cell-specific growth factor and to attenuate vascular disease by inducing endothelial proliferation and regeneration mainly through the endothelial type 2 receptor Flk-1 (Baumgartner I et al., supra). Recent evidence, however, suggests that functional VEGF receptors are expressed in injured arterial walls in cells other than endothelial cells. Therefore, the relative effects of Flt-1-versus Flk-1-mediated action are likely to depend on the relative expression of Flt-1 and Flk-1 in target cells. The present inventor herein demonstrate that Flt-1 was increased in lesional monocytes and medial smooth muscle cells at early stages and in neointimal and medial smooth muscle cells at later stages. Increased Flk-1 expression was noted only at later stages. Taking account of previously reported Flt-1 functions (Barleon B. et al., supra; Ishida A et al., supra; Wang Y H et al., supra; Kim I et al., supra; and Marumo T et al., supra), it is likely that VEGF causes inflammation and migration of vascular smooth muscle cells through Flt-1-mediated signals and thus causes NIH after cuff-induced periarterial injury.

Emerging evidence suggests that hematopoetic stem cells in bone marrow recruit and differentiate into neointimal cells after vascular injury. Sata and colleagues reported that a considerable proportion of neointimal and medial cells were bone marrow-derived progenitor cells that differentiated into smooth muscle cells and endothelial cells in vascular lesions of models of post-angioplasty restenosis, transplant-associated arteriosclerosis, and hyperlipidemia-induced atherosclerosis (Sata M, Saiura A, Kunisato A, Tojo A, Okada S, Tokuhisa T, Hirai H. Makuuchi M, Hirata Y, Nagai R. Hematopoietic stem cells differentiate into vascular cells that participate in the pathogenesis of atherosclerosis. Nat Med. 2002;8:403-9). Flt-1 is an important mediator of stem cell recruitment and mobilization (Luttun A, Tjwa M, Moons L, Wu Y, Angelillo-Scherrer A, Liao F, Nagy JA, Hooper A, Priller J, De Klerck B, Compernolle V, Daci E, Bohlen P, Dewerchin M, Herbert J M, Fava R, Matthys P, Carmeliet G, Collen D, Dvorak H F, Hicklin D J, Carmeliet P. Revascularization of ischemic tissues by PlGF treatment, and inhibition of tumor angiogenesis, arthritis and atherosclerosis by anti-Flt1. Nat Med. 2002;8:831-40). The present inventor herein demonstrate that cells in the neointima or media rarely expressed a marker of bone marrow origin in bone marrow-transplanted mice after cuff placement, indicating a minor contribution of bone marrow-derived cells to neointimal formation in this model.

To gain insight into the mechanism of VEGF-mediated inflammation after cuff placement, the present inventor assessed gene expression of various inflammatory genes. sFlt-1 gene transfer attenuated increased gene expression of inflammatory cytokines, adhesion molecules, chemokines, and chemokine receptors (FIG. 5). These data are consistent with prior reports demonstrating that VEGF induces adhesion molecules (VCAM-1 and ICAM-1) or MCP-1 in endothelial cells in vitro (Kim I et al., supra and Marumo T et al., supra). An essential role of these inflammation-promoting molecules in the development of neointimal hyperplasia after arterial injury has been reported (Egashira K et al, supra, Usui M, Egashira K, Ohtani K, Kataoka C, Ishibashi M, Hiasa K I, Katoh M, Zhao Q, Kitamoto S, Takeshita A. Anti-monocyte chemoattractant protein-1 gene therapy inhibits restenotic changes (neointimal hyperplasia) after balloon injury in rats and monkeys. Faseb J. 2002, Mori E, Komori K, Yamaoka T, Tanii M, Kataoka C, Takeshita A, Usui M, Egashira K, Sugimachi K Essential role of monocyte chemoattractant protein-1 in development of restenotic changes (neointimal hyperplasia and constrictive remodeling) after balloon angioplasty in hypercholesterolemic rabbits. Circulation. 2002;105:2905-10, and Oguchi S, Dimayuga P, Zhu J, Chyu K Y, Yano J, Shah P K, Nilsson J, Cercek B. Monoclonal antibody against vascular cell adhesion molecule-1 inhibits neointimal formation after periadventitial carotid artery injury in genetically hypercholesterolemic mice. Arterioscler Thromb Vasc Biol. 2000;20:1729-36). sFlt-1 gene transfer attenuated increased VEGF and Flt-1 gene expression, indicating that VEGF regulates its activity by an autocrine loop mechanism within diseased arterial wall cells such as smooth muscle cells, endothelial cells, and lesional monocytes. A positive feedback effect of VEGF is supported by prior studies that demonstrated enhanced VEGF production by monocytes through Flt-1 stimulation (Bottomley M J, Webb N J, Watson C J, Holt L, Bukhari M, Denton J, Freemont A J, Brenchley P E. Placenta growth factor (PlGF) induces vascular endothelial growth factor (VEGF) secretion from mononuclear cells and is co-expressed with VEGF in synovial fluid. Clin Exp Immunol. 2000;119:182-8). Therefore, sFlt-1 gene transfer attenuated cuff-induced NIH mainly by suppressing inflammation (monocyte recruitment and activation).

Taken together, VEGF and its receptor signals appear to be essential for the development of early inflammation as well as late NIH after cuff-induced perivascular cuff injury. VEGF is likely to promote NIH by activating and recruiting monocytes and vascular smooth muscle cells. The data shown herein support the notion that VEGF works as a pro-inflammatory and pro-arteriosclerotic factor after cuff-induced periarterial injury.

The present invention provides:

(1) a composition comprising a nucleic acid encoding soluble Flt-1 (sFlt-1) and a pharmaceutically acceptable carrier, wherein said nucleic acid expresses sFlt-1 in an amount effective to inhibit or treat inflammation of vessel wall and/or formation of neointimal hyperplasia;

(2) the composition of (1), wherein the nucleic acid is inserted in a vector;

(3) the composition of (2), wherein the vector is selected from the group consisting of a plasmid, an adenovirus vector, and a Hemagglutinating virus of Japan envelope (HVJ-E) vector;

(4) the composition of (2), wherein the vector is a eukaryotic expression plasmid;

(5) the composition of any one of (1) to (4), wherein the nucleic acid encodes a polypeptide comprising an amino acid sequence of SEQ ID NO: 2;

(6) the composition of any one of (1) to (5), wherein the nucleic acid encodes a polypeptide comprising an amino acid sequence of SEQ ID NO: 2 which has one or more amino acid substitution, deletion, addition, and/or insertion, wherein said polypeptide is functionally equivalent to and has at least 65% identity to a polypeptide comprising amino acid sequence of SEQ ID NO: 2;

(7) the composition of any one of (1) to (6), wherein the amount effective to inhibit inflammation of vessel wall and/or formation of neointimal hyperplasia is between about 0.0001 mg and 100 mg per day per patient;

(8) the composition of any one of (1) to (7), wherein the composition is administered to a patient intramuscularly;

(9) the composition of any one of (1) to (8), wherein the composition is administered to a patient with risk of post coronary intervention restenosis, atherosclerosis, arteriosclerosis, or edema;

(10) the composition of (9), wherein the patient is a hypercholesterolemia patient;

(11) use of a nucleic acid encoding soluble Flt-1 (sFlt-1) for the production of a pharmaceutical composition for inhibiting or treating inflammation of vessel wall and/or formation of neointimal hyperplasia;

(12) the use of (11), wherein the nucleic acid is inserted in a vector;

(13) the use of (12), wherein the vector is selected from the group consisting of a plasmid, an adenovirus vector, and a Hemagglutinating virus of Japan envelope (HVJ-E) vector;

(14) the use of (12), wherein the vector is a eukaryotic expression plasmid;

(15) the use of any one of (11) to (14), wherein the nucleic acid encodes a polypeptide comprising an amino acid sequence of SEQ ID NO: 2;

(16) The use of any one of (11) to (15), wherein the nucleic acid encodes a polypeptide comprising an amino acid sequence of SEQ ID NO: 2 which has one or more amino acid substitution, deletion, addition, and/or insertion, wherein said polypeptide is functionally equivalent to and has at least 65% identity to a polypeptide comprising amino acid sequence of SEQ ID NO: 2;

(17) the use of any one of (11) to (16), wherein the composition is administered at a dose between about 0.0001 mg and 100 mg per day per patient;

(18) the use of any one of (11) to (17), wherein the composition is administered to a patient intramuscularly;

(19) the use of any one of (11) to (18), wherein the composition is administered to a patient with risk of post coronary intervention restenosis, atherosclerosis, arteriosclerosis, or edema;

(20) the use of (19), wherein the patient is a hypercholesterolemia patient;

(21) a method for inhibiting or treating inflammation of vessel wall and/or formation of neointimal hyperplasia, comprising administration of a nucleic acid encoding soluble Flt-1 (sFlt-1) to a patient in need thereof,

(22) the method of (21), wherein the nucleic acid is inserted in a vector;

(23) the method of (22), wherein the vector is selected from the group consisting of a plasmid, an adenovirus vector, and a Hemagglutinating virus of Japan envelope (HVJ-E) vector;

(24) the method of (22), wherein the vector is a eukaryotic expression plasmid;

(25) the method of any one of (21) to (24), wherein the nucleic acid encodes a polypeptide comprising an amino acid sequence of SEQ ID NO: 2;

(26) the method of any one of (21) to (25), wherein the nucleic acid encodes a polypeptide comprising an amino acid sequence of SEQ ID NO: 2 which has one or more amino acid substitution, deletion, addition, and/or insertion, wherein said polypeptide is functionally equivalent to and has at least 65% identity to a polypeptide comprising amino acid sequence of SEQ ID NO: 2;

(27) the method of any one of (21) to (26), wherein the nucleic acid is administered at a dose between about 0.0001 mg and 100 mg per day per patient;

(28) the method of any one of (21) to (27), wherein the nucleic acid is administered intramuscularly;

(29) the method of any one of (21) to (28) wherein the patient has risk factors for post coronary intervention restenosis, atherosclerosis, arteriosclerosis, or edema; and

(30) the method of (29), wherein the patient is a hypercholesterolemia patient.

The present invention is described in more detail below.

Polynucleotides

As used herein, “a soluble Flt-1 (sFlt-1) gene” means a polynucleotide or nucleic acid that encodes and expresses an sFlt-1 protein. Such a polynucleotide or nucleic acid may be DNA or RNA It can be obtained by isolation from a natural source or by synthesis.

As used herein, an “isolated polynucleotide or nucleic acid” is a polynucleotide or nucleic acid removed from its original environment (e.g., the natural environment if naturally occurring) and thus, altered by the “hand of man” from its natural state.

The term therefore covers, for example, (a) a DNA fragment of a naturally occurring genomic DNA molecule free of the coding sequences that naturally flank the nucleic acid (i.e., sequences located at the 5′ and 3′ ends of the nucleic acid) in the genomic DNA in the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in random, uncharacterized mixtures of different DNA molecules, transfected cells, or cell clones, e.g., as these occur in a DNA library such as a cDNA or genomic DNA library.

A naturally-occurring nucleic acid can be derived from mammals including mice, rats, and humans. The known human sFlt-1 gene (Kendall R L et al. 1993 supra, GenBank accession number U01134), as shown in SEQ ID NO: 1, and mouse sFlt-1 gene (GenBank accession number D88690), as shown in SEQ ID NO: 3, can be used. An sFlt-1 gene used in this invention can be synthesized based on its known sequence. For example, it is possible to clone the cDNA of sFlt-1 by performing a RT-PCR reaction on mRNA derived from a suitable source using a suitable DNA portion as a PCR primer. Such cloning can easily be performed by a person skilled in the art according to a reference, such as Maniatis T. et al., Molecular Cloning 2nd Ed., Cold Spring Harbor Laboratory Press (1989).

So long as a polypeptide expressed from the sFlt-1 gene is functionally equivalent to sFlt-1, the sFlt-1 gene may have a partial deletion, substitution, or insertion of one or more nucleic acid, or may have other nucleotide sequence ligated therewith at the 5′ terminus and/or 3′ terminus thereof. Here, “sFlt-1 activity” means that the activity to bind to VEGF but not to result in signal transduction.

Herein, the phrase “functionally equivalent” means that the subject polypeptide retains a biologically significant activity that is characteristic of sFlt-1. Examples of biologically significant activities of sFlt-1 include VEGF inhibitory activities that inhibit inflammation and migration of vascular smooth muscle cells. Accordingly, the present invention includes polynucleotides comprising a nucleotide sequence encoding a protein having the amino acid sequence of SEQ ID NO: 1, in which one or more amino acids are substituted, deleted, inserted and/or added, so long as the resulting protein retains sFlt-1 activity. Moreover, the present invention also includes polynucleotides that hybridize under stringent conditions with a DNA consisting of the nucleotide sequence of SEQ ID NO: 1, so long as the resulting polynucleotide encodes a protein that are functionally equivalent to sFlt-1. The determination of sFlt-1 can be conducted by methods well known to those skilled in the art, such as VEGF-binding assay as described in Duan, D-S, R. et al., (1991) J. Biol. Chem., 266, pp. 413-418, and mitogen inhibition assay as described in WO94/21679.

Polynucleotides of the present invention can be obtained by methods well known to those skilled in the art. Examples of such methods include site-directed mutagenesis (Kramer, W. and Fritz, H J (1987) Methods in Enzymol. 154:350-367), hybridization technique (E. M. Southern, J. Mol. Biol. 1975, 98: 503-517) and polymerase chain reaction (PCR) technique (R. K. Saiki et al., Science 1985, 230: 1350-1354; R. K. Saiki et al., Science 1988, 239: 487-491). More specifically, those skilled in the art can generally isolate polynucleotides highly homologous to the polynucleotide shown in SEQ ID NO: 1 from other animals, using the polynucleotide shown in SEQ ID NO: 1 or a part thereof as probes or using the oligonucleotide which specifically hybridizes with the polynucleotide shown in SEQ ID NO: 1 as primers. Furthermore, polynucleotides that can be isolated by hybridization techniques or PCR techniques and that hybridize with polynucleotides shown in SEQ ID NO: 1 are also included in the polynucleotides of the present invention. Examples of such polynucleotides include polynucleotides disclosed in WO94/21679.

Hybridization reactions to isolate polynucleotides as described above are preferably conducted under stringent conditions. Hybridization may be performed with buffers that permit the formation of a hybridization complex between nucleic acid sequences that contain some mismatches. At high stringency, hybridization complexes will remain stable only where the nucleic acid molecules are almost completely complementary. Many factors determine the stringency of hybridization, including G+C content of the cDNA, salt concentration, and temperature. For example, stringency may be increased by reducing the concentration of salt or by raising the hybridization temperature. Temperature conditions for hybridization and washing greatly influence stringency and can be adjusted using melting temperature (Tm). Tm varies with the ratio of constitutive nucleotides in the hybridizing base pairs, and with the composition of the hybridization solution (concentrations of salts, formamide and sodium dodecyl sulfate). In solutions used for some membrane based hybridizations, addition of an organic solvent, such as formamide, allows the reaction to occur at a lower temperature. Accordingly, on considering the relevant parameters, one skilled in the art can select appropriate conditions to achieve a suitable stringency based experience or experimentation.

Examples of stringent hybridization conditions includes conditions comprising: 65° C., 2×SSC, 0,1% SDS and those having a stringency equivalent to the conditions. In general the higher the temperature, the higher is the homology between two strands hybridizing at equilibrium. Polynucleotides isolated under higher stringency conditions, such as described above, are expected to encode a polypeptide having a higher homology at the amino acid level to the amino acid sequence shown in SEQ ID NO: 2. In this context, “high homology” means an identity of at least 65% or more, more preferably 70% or more, still more preferably 80%, further more preferably 90% or more, and most preferably 95% or more, in the whole amino acid sequence.

Polypeptides

An sFlt-1 protein encoded by the nucleic acids as described above includes human sFlt-1 as shown in SEQ ID NO: 2, mouse sFlt-1 as shown in SEQ ID NO: 4, and its variants. The variants are preferably encoded by the nucleotide sequence having at least 65% identity to human sFlt-1 gene. More preferably, the variant is at least 65%, 70%, 75%, 80%, 85%, 90%, 95%, 96%, 97%, 98%, 99%, or more, identical to the nucleotide sequence of human sFlt-1 gene. For example, when an isolated polynucleotide of the present invention, e.g., SEQ ID NO: 1 is longer than or equivalent in length to a prior art sequence, the comparison is made with the full length of the inventive sequence. Alternatively, when the isolated polynucleotide of the present invention is shorter than the prior art sequence, the comparison is made to a segment of the prior art sequence of the same length as that of the inventive sequence (excluding any loop required by the homology calculation).

The determination of percent identity between two sequences can be accomplished using any conventional mathematical algorithm, such as the BLAST algorithm by Karlin and Altschul (S. Karlin and S. F. Altschul, Proc. Natl. Acad. Sci. USA. 1990, 87: 2264-2268; S. Karlin and S. F. Altschul, Proc. Natl. Acad. Sci USA. 1993, 90: 5873-5877). The BLAST algorithm is incorporated into the BLASTN and BLASTX programs of Altschul et al. (S. F. Altschul et al., J. Mol. Biol. 1990, 215: 403). When a nucleotide sequence is analyzed according to BLASTN, suitable parameters include, for example, a score=100 and word length=12. On the other hand, suitable parameters for the analysis of amino acid sequences by BLASTX include, for example, a score=50 and word length=3. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. When utilizing BLAST, Gapped BLAST, and PSI-Blast programs, the default parameters of the respective programs (e.g., XBLAST and NBLAST) are preferably used. However, one skilled in the art can readily adjust the parameters to suit a particular purpose. Specific procedures for such analysis are known in the art (See, for example, the BLAST website of the National Center for Biotechnology Information located on the worldwide web at www.ncbi.nlm nih.gov). Another example of a mathematical algorithm that may be utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

Polypeptides having amino acid sequences modified by deleting, adding and/or replacing one or more amino acid residues of a certain amino acid sequence, have been known to retain the original biological activity (Mark, D. F. et al., Proc. Natl. Acad. Sci. USA (1984) 81, 5662-5666, Zoller, M. J. & Smith, M., Nucleic Acids Research (1982) 10, 6487-6500, Wang, A. et al., Science 224, 1431-1433, Dalbadie-McFarland, C et al., Proc. Natl. Acad. Sci USA (1982) 79, 6409-6413).

The number of amino acids that are mutated by substitution, deletion, addition, and/or insertion is not particularly restricted. Normally, it is 10% or less, preferably 5% or less, and more preferably 1% or less of the total amino acid residues.

Amino acid substitutions may be made at one or more predicted, preferably nonessential amino acid residues. A “nonessential” amino acid residue is a residue that can be altered from the wild-type sequence of a protein (e.g., the sequence shown in SEQ ID NO: 2) without altering the biological activity, whereas an “essential” amino acid residue is required for biological activity. An amino acid is preferably substituted for a different amino acid(s) that allows the properties of the amino acid side-chain to be conserved. Accordingly, a “conservative amino acid substitution” is a replacement in which the amino acid residue is replaced with an amino acid residue having a chemically similar side chain. Groups of amino acid residues having similar side chains have been defined in the art. These groups include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Vectors

An sFlt-1 gene of the present invention may be incorporated into various vectors. Any vectors can be used so long as they permit the in vivo expression of the gene. Examples of vectors includes plasmids, liposomes, and viral vectors.

As plasmid vectors, eukaryotic plasmids are preferably used, including pCAGGS (Gene 108:193-200(1991)), pBK-CMV, pcDNA3.1, and pZeoSV (Invitrogen, Stratagene). In such expression vectors, a gene of this invention can be operably linked to promoter/enhancer elements. The promoter/enhancer elements may be selected to optimize for the in vivo expression of the gene. The promoter may be inducible or constitutive, and, optionally, tissue-specific. Promoters isolated from the genome of viruses that grow in mammalian cells, such as vaccinia virus 7.5 K, SV40, HSV, adenoviruses MLP, MMTV, LTR and CMV promoters, may be used.

Alternatively, an sFlt-1 gene of the present invention can be encapsulated into any known liposome made of lipid bilayer such as an electrostatic liposome. A liposome containing an sFlt-1 gene of the present invention can be fused to viruses such as inactivated Sendai virus (Hemagglutinating virus of Japan: HVJ). The HVJ-liposome has very high fusing activity with the cell membrane as compared to the conventional liposomes. In particular, the Z strain (available from ATCC) is preferred as the HVJ strain, but other HVJ strains (for example, ATCC VR-907 and ATCC VR-105) may also be used.

Viral vectors can also be used. Viral vectors can be DNA viruses or RNA viruses. Examples of the viral vectors include detoxified retrovirus, adenovirus, adeno-associated virus, herpes virus, vaccinia virus, poxvirus, poliovirus, Sindbis virus, Hemagglutinating virus of Japan envelope (HVJ-E, Sendai virus), SV40, and human immunodeficiency virus (HIV). Among the above viral vectors, the efficiency of infection of adenovirus is known to be much higher than that of other viral vectors. In this regard, an adenovirus vector system can be preferably used.

The above-described vectors and liposomes can be prepared according to a known method (Supplement of Experimental Medicine, Basic Technology in gene therapy, Yodosha (1996); Supplement of Experimental Medicine, Experimental Methods in Gene Introduction and Expression Analysis, Yodosha (1997); Handbook for Development and Research of Gene Therapy, Japan Society of Gene Therapy ed., NTS (1999), J.Clin.Invest. 93:1458-1464(1994); Am.J.Physiol. 271:R1212-1220 (1996), etc.).

Gene Therapy Agents

A vector carrying an sFlt-1 gene of the present invention can be formulated into an appropriate gene therapy agent. The term “gene therapy agent” used herein means a pharmaceutical composition used as a dosage form for gene therapy. The composition may vary depending on administration regimens described below (e.g. liquids). For example, an injection may be prepared by dissolving the gene into an appropriate solvent (a buffer such as PBS, physiological saline, sterile water, etc.). The injection liquid may then be filter-sterilized with filter as needed and then filled into sterilized containers. Conventional carriers and so on may be added to the injection. Liposomes, such as HVJ-liposome, may take the form of suspensions, frozen formulations, centrifugation-concentrated frozen formulations, and the like.

Gene therapy agents of the present invention comprise a vector carrying an sFlt-1 gene, so that sFlt-1 is expressed in an amount effective to inhibit or treat inflammation of vessel walls or to inhibit formation of NIH. Such inhibitory effects can be determined as described in the examples below. For example, inflammation-inhibitory effects can be determined by measuring the number of Mac3-positive monocytes (see Examples 2, 3, and 4, FIG. 3E). NIH formation-inhibitory effects can be determined by measuring intimal area, intima/media ratio, and % stenosis (see Example 4, FIG. 3B, C, and D). The expression levels of VEGF, Flt-1, CCR1, IL-6, CCR2, MCP-1, Flt-1, CXCR2, eotaxin, VCAM-1, or ICAM-1 can also be used as an indicator for inflammation- and NIH formation-inhibitory effects (see Examples 3 and 5). For the VEGF level VEGF₁₈₈ and VEGF₁₆₄ or their corresponding isoforms, except VEGF₁₂₁ or its corresponding isoforms, should be determined. The expression level can be measured for an mRNA level or protein level using a known method such as Northern blotting and Western blotting (e.g., Maniatis T. et al., supra). Alternatively, NIH can be measured by cardio angiography (CAG) or intravascular ultrasounds (VUS). The data obtained by these methods can be compared with the data obtained for normal NIH-free sites to thereby determine NIH formation-inhibitory effects.

Gene Transfer Methods

A gene therapy agent of the present invention may be introduced into target cells or tissues of patients by in vivo methods or ex vivo methods. In vivo methods permit direct introduction of the gene therapy agent into the body. In ex vivo methods, certain cells are removed from human, the gene therapy agent is introduced into the cells, and the resulting cells are returned into the body thereafter (Nikkei Science, April 1994 issue pp. 20-24; Monthly Yakuji, 36(1): 23-48 (1994); Supplement To Experimental Medicine 12(15) (1994); Handbook for Development and Research of Gene Therapy, NTS (1999)). According to the present invention, in vivo methods are preferred.

Illustrative methods of gene transfer into cells include the lipofection method, calcium phosphate co-precipitation method, DEAE-dextran method, direct DNA introduction methods using micro glass tubes, and the like.

Exemplary methods of gene transfer into tissues include internal type liposome method, electrostatic type liposome method, HVJ-liposome method, improved HVJ-liposome method (HVJ-AVE liposome method), receptor-mediated gene introduction, particle gun method, naked-DNA method, method of introduction with positively-charged polymers, etc.

To enhance transgene expression, electroporation may be applied following the gene transfer. Microinjection or viral vectors can also be used for efficient gene transfer or transgene expression.

Proper methods and sites for administration adequate for the disease or symptom to be treated are selected for the gene therapy of this invention. A gene therapy agent of this invention can be administered parenterally, preferably intramuscularly, to the site of vascular injury.

A dosage of an agent of this invention varies depending on the age, gender, and symptoms of the patient, but sFlt-1 gene can be administered at a dose of about 0.0001 mg to about 100 mg, preferably about 0.001 to about 10 mg per day per adult patient. When the HVJ-liposome form is used, a gene of this invention can be administered in a range of about 1 to about 4000 μg, preferably about 10 to about 400 μg per adult patient.

The therapeutic agent of this invention may be administered once every few days or every few weeks, or once per week. Frequency of administration is to be selected depending on the symptoms of the patients. In compliance with the object of the treatment, plural administration is suitable.

The gene therapy of the present invention is effective for inhibiting inflammation of vessel wall and/or formation of NIH. Inflammation of vessel wall and formation of NIH are symptoms observed after vascular injury caused by post coronary intervention restenosis, atherosclerosis, or arteriosclerosis. The gene therapy of the present invention can be applied to patients with risk of these diseases. The risk factors for these diseases include hypercholesterolemia.

Any publications referenced herein are hereby incorporated by reference in this application in order to more fully describe the state of the art to which the present invention pertains.

### BRIEF DESCRIPTION OF THE DRAWINGS

FIG. 1 shows VEGF expression in cuffed femoral artery. A, A photograph showing time course of VEGF mRNA levels. Expression of arterial VEGF and β-actin mRNA after cuff placement. mRNA levels were assessed at the indicated times. This is a representative assay from five separate experiments. B, Densitometric analysis of data in A. Expression of VEGF mRNA in each sample was normalized by β-actin mRNA expression in the same sample. N=5 for each bar. *P<0.01 vs control intact artery. C, Photographs showing cross-sections of intact or cuffed femoral arteries were stained immunohistochemically against VEGF, VEGF receptor 1 (Flt-1), VEGF receptor 2 (Flk-1) or vWF 7 or 21 days after cuff placement. Bar indicates 50 μm.

FIG. 2 shows immunofluorescence staining of VEGF receptors, monocytes, and α-SM actin in cuffed femoral artery. A, Micrographs of cuffed femoral arteries stained with Flt-1 (VEGF-R1, green) and α-SM actin (red), with Flk-1 (VEGF-R2, green) and α-SM actin (red), and with Mac-3 (green) and Flt-1 (VEGF-R1, red) 7 days after cuff placement. Bar indicates 10 μm. B, Micrographs of cuffed femoral arteries stained with Flt-1 (VEGF-R1) and α-SM actin, and Flk-1 (VEGF-R2) and α-SM actin in the cuffed femoral arteries 21 days after cuff placement. Single fluorescence-positive cells were stained green or red, whereas double-positive cells were stained yellow. Scale bar indicates 10 μm FIG. 3 shows histopathology of cuffed femoral artery. A, Photographs showing time course of cuff injury-induced NIH and effect of sFit-1 gene transfer. Micrographs of cross-sections of control (intact) and cuffed arteries stained with van Gieson Elastica (vGE) on days 3, 7, and 21 are shown. Scale bar indicates 100 μm. B, C, and D, Effects of sFlt-1 gene transfer on neointimal thickening (B), intima/media ratio (C), % stenosis (D) 21 days after cuff placement. E, Effects of sFlt-1 gene transfer on inflammatory and proliferative changes 7 days after cuff placement. *P<0.01 vs control and sFlt-1 group.

FIG. 4 shows contribution of bone marrow-derived cells in the development of the neointima after cuff placement. Micrograph of cross-section stained with LacZ 21 d after cuff placement in bone marrow-transplanted mice. There were no LacZ-positive cells in the neointima or media. Lac Z-positive cells were present only in the adventitia (arrows). This is a representative sample from six animals. Scale bar indicates 50 μm.

FIG. 5 is a photograph showing effects of sFlt-1 gene transfer on chemokines and chemokine receptors (MCP-1, CCR2, RANTES, CCR1, MIP-1α, CXCR2, eotaxin, MIP-2), adhesion molecules (ICAM-1, VCAM-1), cytokines (IL-6, TGF-β), VEGF, and Flt-1 in cuffed femoral arteries. Data are expressed as the ratio of each mRNA to the corresponding GAPDH mRNA. *P<0.01 versus control site; †P<0.01 versus empty plasmid group; NS indicates not significant. N=5-6.

### BEST MODE FOR CARRYING OUT THE INVENTION

The present invention will now be specifically explained with reference to the following examples. It should be noted, however, that the present invention is not limited by these examples in any way.

General Methods

The following methods are general to all examples that follow.

Expression Vector

The 3.3-kb mouse sFlt-1 gene (Genbank accession number D88690; nucleotide and amino acid sequences are shown in SEQ ID NO: 3 and SEQ ID NO: 4, respectively) was obtained from a mouse lung cDNA library (Kondo K, Hiratsuka S, Subbalakshmi E, Matsushime H, Shibuya M. Genomic organization of the flt-1 gene encoding for vascular endothelial growth factor (VEGF) receptor-1 suggests an intimate evolutionary relationship between the 7-Ig and the 5-Ig tyrosine kinase receptors. Gene. 1998; 208:297-305) and cloned into the multicloning site, the BamH1(5′) and Not1(3′) sites of the eukaryotic expression vector plasmid cDNA3 (Invitrogen). In this plasmid, gene expression is controlled by the cytomegalovirus immediate early enhancer/promoter.

Experimental Animals

Apolipoprotein E knockout mice (8-10 week old) with a genetic background of C57BL/6J were purchased from Jackson Laboratory (Bar Harbor, Me.) and fed with commercial standard chow. Placement of cuff and gene transfer were performed as previously described (Zhao Q et al., supra; and Egashira K et al., supra). A non-constrictive polyethylene cuff (1.5-mm ng; PE20, 0.38-mm inner diameter, 1.09-mm outer diameter) was placed loosely around the left femoral artery. Either empty plasmid or sFlt-1 plasmid (300 μg/100 μl PBS) was injected into the right femoral muscle using a 27-gauge needle immediately after and 10 days thereafter. To enhance transgene expression, these animals received electroporation at the injected site immediately after injection. Six electric pulses of 100 V for 50 ms were applied with the use of an electric pulse generator CUY21 (BTX).

Cuff placement was also performed in wild-type mice with a genetic background of C57BL/6J whose bone marrow was replaced with that of ROSA26 mice, which ubiquitously expresses β-galactosidase (LacZ) (Sata M et al., supra). Lethally-irradiated wild-type mice received 1×10⁶ bone-marrow cells from a ROSA26 mouse. Four weeks after bone-marrow transplantation, a cuff was placed around the left femoral artery. The femoral artery was excised and stained with X-gal solution for 7 h, then further fixed in 4% paraformaldehyde.

Histopathology and Immunohistochemistry

Mice were anesthetized with pentobarbital, and the femoral artery was harvested, fixed overnight in 3.7% formaldehyde in phosphate buffered saline, and paraffin-embedded (Egashira K et al., supra). Serial cross sections (5 μm thick) throughout the entire length of the cuffed femoral artery were used for histologic analysis. Cryosections were made from two mice in each condition. All sections were routinely stained with hematoxylin-eosin (HE) or van Gieson. Mac-3 (PharMingen) staining was used to detect monocytes/macrophages. Proliferating cell nuclear antigen (Santa Cruz Biotech, Santa Cruz, Calif.) was used to detect vascular proliferation. An antibody against von Willebrand factor (vWF; Sigma Chemical Co., St. Louis, Mo.) was used to mark endothelial cells. Indirect immunofluorescence double-staining with matched primary and fluorescein conjugated secondary antibodies was used to stain for colocalization with VEGF receptors in smooth muscle cells or monocytes: Rabbit anti-mouse Flt-1 (Santa Cruz Biotech), rabbit anti-mouse Flk-1 (Santa Cruz Biotech), rat anti-mouse Mac-3, anti-smooth muscle actin (α-SMA) (Boehringer Mannheim), anti-rabbit IgG conjugated with FITC or rhodamine, and anti-rat IgG conjugated with FITC or rhodamine (Santa Cruz Biotech).

Quantificafion of Intimal Lesions in Sections of Cuffed Femoral Artery

Ten equally-spaced cross-sections were examined in all mice to quantify intimal lesions. Using image analysis software, the total cross-sectional medial area was measured between the external and internal elastic lamina; the total cross-sectional intimal area was measured between the endothelial cell monolayer and the internal elastic lamina.

Reverse Transcription-Polymerase Chain Reaction and RNAse Protection Assay

RNA was prepared from the pooled samples (n=5-7 for each group) using TRIzol reagent (Gibco-BRL). First-strand DNA was synthesized using reverse transcriptase with random hexamers from 1 μg total RNA in a 20-μl reaction volume according to the manufacturer's protocol (GeneAmp RNA PCR Kit; Perkin-Elmer). Ten percent of the resulting reverse transcription (RT) product was amplified using 25 μl polymerase chain reaction (PCR). Primers used for amplification of VEGF were 5′-GGA TCC ATG AAC TTT CTG CT-3′ (SEQ ID NO: 5) and 5′-GAA TTC ACC GCC TCG GCT TGT C-3′ (SEQ ID NO: 6) with expected sizes of 654 bp, 582 bp, and 450 bp for the three VEGF isoforms (VEGF 188, 164, and 121, respectively). Primers for the internal control, β-actin, were 5′-ATG GAT GAC GAT ATC GCT-3′ (SEQ ID NO: 7) and 5′-ATG AGG TAG TCT GCT AGG T-3′ (SEQ ID NO: 8) with an expected product of 550 bp. PCR products were separated by 2% agarose gel electrophoresis, visualized using ethidium bromide, photographed, and analyzed by scanning densitometry.

RNAse protection assays were performed using 5 μg of total RNA with two custom template sets according to the manufacturer's protocol (PharMingen). After RNAse digestion, protected probes were resolved on denaturing polyacrylamide gels and quantified using a BASS-3000 system (Fuji Film). The value of each hybridized probe was normalized to that of the internal controls, L32 and GAPDH, included within each template set.

Statistical Analysis

Data are expressed as the mean±SE. Statistical analysis of differences was compared by analysis of variance. Post hoc analyses were performed using Bonferroni's correction for multiple comparison tests. A P level of less than 0.05 was considered to be statistically significant.

Results

### EXAMPLE 1

### Plasma Lipid Levels

Plasma total cholesterol, triacylglycerol, and high density lipoprotein-cholesterol levels were determined with commercially available kits (Wako Pure Chemicals, Osaka, Japan). There were no statistically significant differences in serum total cholesterol and triacylglycerol levels among the three groups; the control group, the empty plasmid group, and the sFlt-1 group. Total cholesterol and triacylglycerol levels were 503±11 and 38±6 mg/dL in the control group, 512±16 and 40±5 mg/dL in the empty plasmid group, and 497±10 and 39±3 mg/dL in the sFlt-1 group.

### EXAMPLE 2

### In Vivo Plug Assay

An in vivo matrigel plug assay was used to determine the effect of sFlt-1 gene transfer on VEGF activity (Zhao Q et al., supra; and Egashira K et al., supra). Matrigel matrix alone (300 μL) or mixed with recombinant VEGF protein (100 ng/mL) was injected subcutaneously into the flanks of C57BL/6J mice. The matrigels were then removed 7 or 14 days after injection, and angiogenesis and inflammation were examined by histopathologic analysis.

Seven days after cuff placement, there were significant angiogenic (number of CD31 positive cells/mm², 380±29) and inflammatory (number of Mac3-positive cells/mm²; 87±10/mm²) reactions in the matrigel plugs containing recombinant VEGF protein compared to matrigel alone (8±3 and 5±2, respectively). Soluble Flt-1 gene transfer, but not injection of an empty plasmid, suppressed both the angiogenic (11±5/mm²) and inflammatory (6±3/mm²) reactions to VEGF to a level similar to that of matrigel plugs without VEGF.

### EXAMPLE 3

### Increased Expression of VEGF mRNA and Immunoreactivity

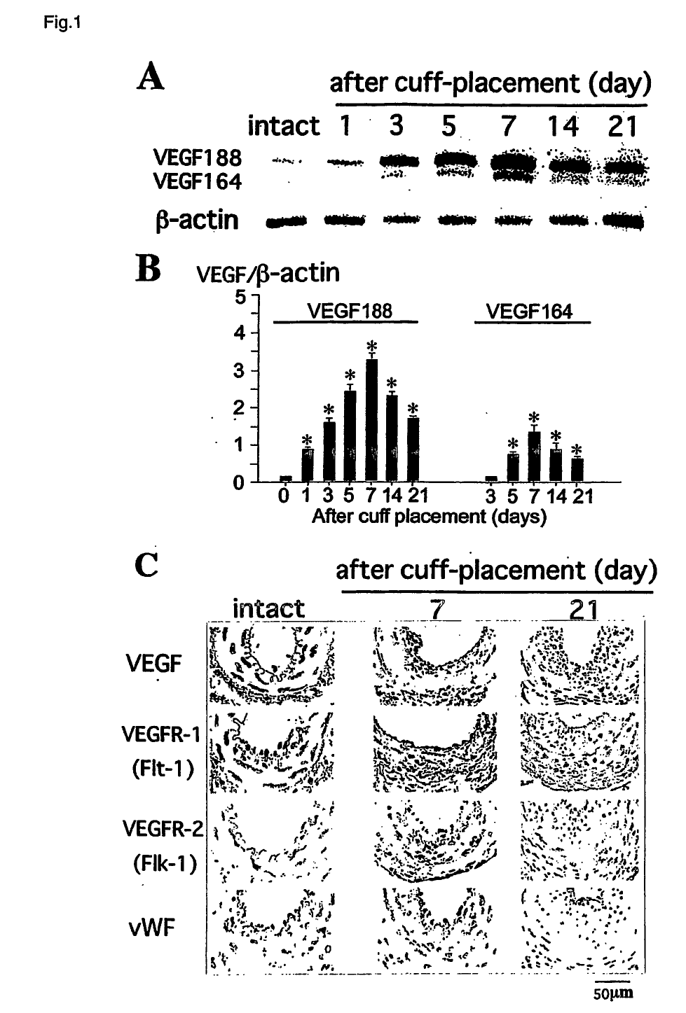

The mRNA levels of two VEGF isoforms (188 and 164) markedly increased after cuff placement whereas they were undetectably low in control intact artery (FIGS. 1A, B). Peak expression was observed on day 7. VEGF 121 mRNA was undetectable before and after cuff placement.

Immunohistochemical staining indicated that compared to faint staining in the control artery, VEGF increased in the vicinity of inflammatory lesions (mononuclear cell infiltration) in the intima and adventitia on day 7 and in cells of three layers of cuffed artery on day 21 (FIG. 1C). The endothelial layer, as detected by vWF staining, was preserved before and after cuff placement (FIG. 1C).

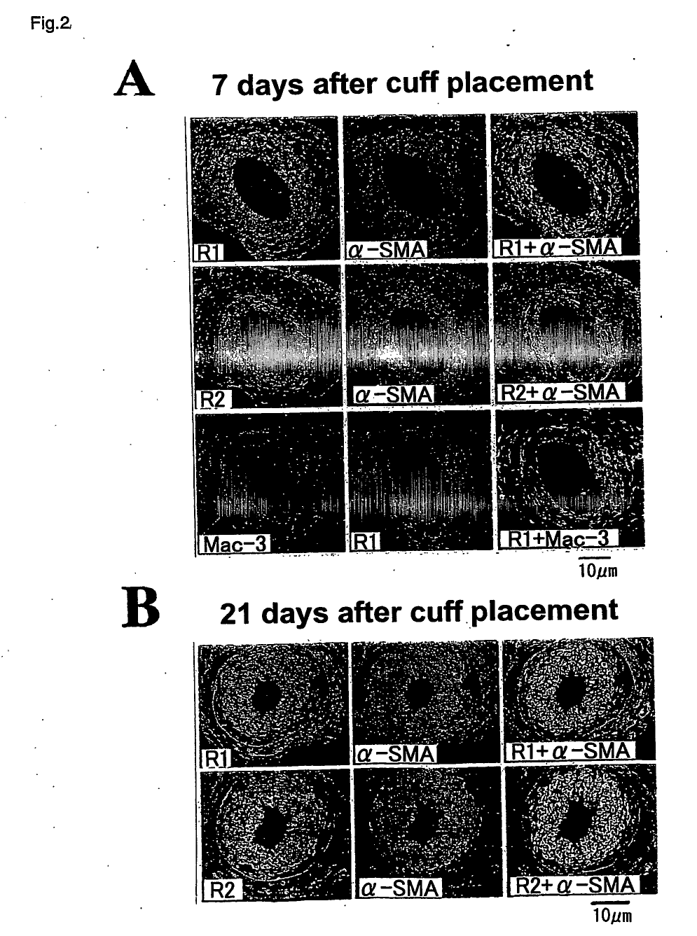

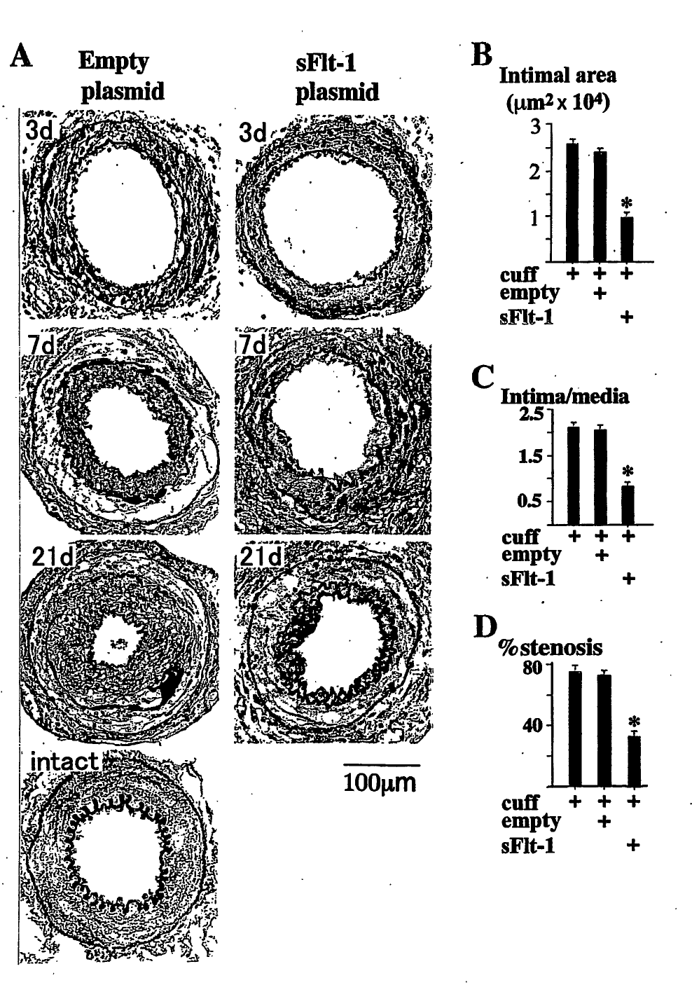

Flt-1 was undetectable except in endothelial layers in control intact arteries, but was drastically increased in the intima, media, and adventitia 7 and 21 days after cuff placement (FIG. 1C). VEGFR-2 (Flk-1) was not increased on day 7, but did increase on day 21. To localize VEGF receptors, immunofluorescent double-staining was performed (FIG. 2). On day 7, α-SM actin-positive cells in the media and neointima expressed very little Flk-1, whereas they did express Fit-1 (FIG. 2A). Mac-3 positive cells recruited to the neointima, media, and adventitia expressed Flt-1, but not Flk-1. Also, some α-SM actin-positive cells in the adventitia (possibly adventitial myofibroblasts) expressed Flt-1. On day 21, most α-SM actin-positive cells in the neointima and media expressed both VEGF receptors (FIG. 2B).

### EXAMPLE 4

### Time Course of Development of Neointimal Hyperplasia

Mice were killed on days 1, 3, 7, 14, and 21. As published (Lardenoye J H et al., supra; Egashira K et al., supra; Wu L et al., supra; and Moroi M, Zhang L, Yasuda T, Virmani R, Gold H K, Fishman M C, Huang P L. Interaction of genetic deficiency of endothelial nitric oxide, gender, and pregnancy in vascular response to injury in mice. J Clin Invest. 1998;101:1225-32), within 7 days of cuff placement, mononuclear leukocytes, most of which were Mac3-positive monocytes, were recruited into the adventitia, media, and intima (FIG. 2). After day 7, neointimal lesions developed and became thick over time (FIG. 3A). Monocyte infiltration declined spontaneously and α-SM actin-positive cells appeared predominantly in the neointima. On day 21, significant NIH with luminal stenosis developed. The cells in the neointima consisted predominantly of α-SM actin-positive cells.

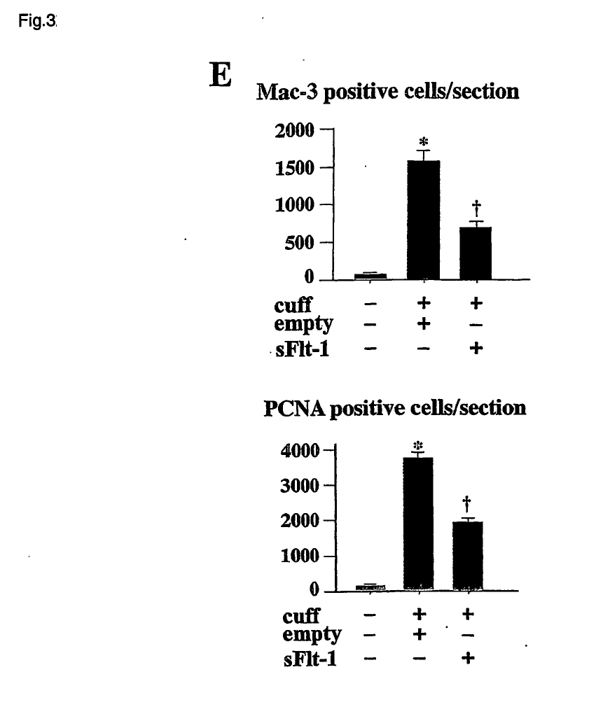

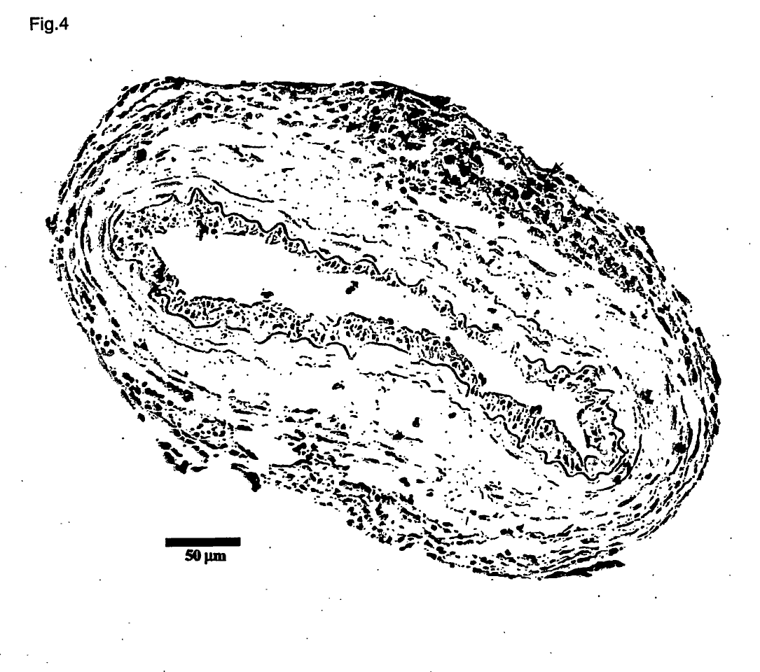

To determine whether bone marrow-derived progenitor cells contribute to neointimal formation, the present inventor used bone marrow-transplanted mice whose bone marrow expressed β-galactosidase. β-galactosidase (LacZ) of normal and cuffed artery was stained 21 days after cuff placement. LacZ-positive cells were rarely observed in the neointima or media (FIG. 4). Some mononuclear leukocytes recruited into in the adventitia were positive for LacZ.

### EXAMPLE 5

### Soluble Flt-1 Gene Transfer Attenuates Cuff-Induced Neointimal Hyperplasia

There was markedly less inflammation (Mac3-positive cells) and proliferation (the PCNA index) in sFlt-1-transfected mice than in empty plasmid-transfected mice at day 7 (FIG. 3E). sFlt-1 gene transfer significantly reduced NIH (increases in neointimal area, intima/media ratio, and luminal stenosis) 21 days after cuff placement (FIGS. 3A, B, C, and D).

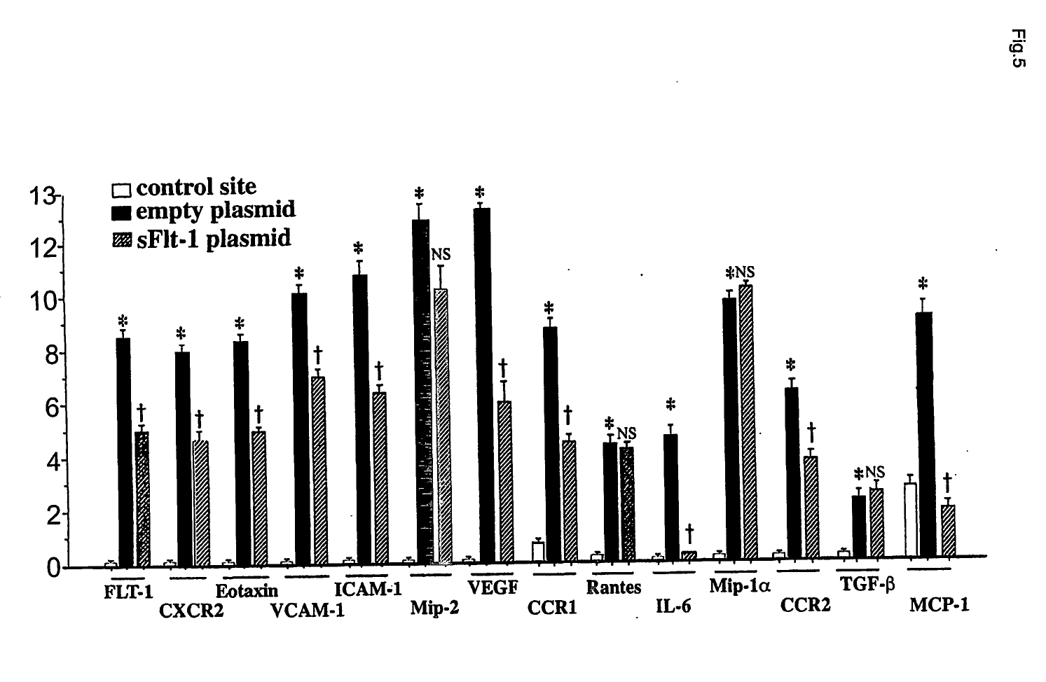

Gene expression of a battery of inflammatory cytokines, chemokines, and chemokine receptors were examined by RNAse protection assays 7 days after cuff placement (FIG. 5). Gene expression was upregulated after cuff placement. sFlt-1 gene transfer did not affect gene expression of RANTES, MIP-1α, TGF-β, MIP-2, but prevented or attenuated the increased gene expression of CCR1, IL-6, CCR2, MCP-1, Flt-1, CXCR2, eotaxin, VCAM-1, ICAM-1, and VEGF.

### INDUSTRIAL APPLICABILITY

The present invention has potentially significant clinical implications. Blockade of VEGF by sFlt-1 gene transfer can be an attractive anti-VEGF therapy for inflammatory vascular diseases and other inflammatory disorders. In particular, this gene therapy is useful to inhibit the development of NIH after vascular injury caused by post coronary intervention restenosis, atherosclerosis, arteriosclerosis, or edema. Therefore, the compositions and methods of the present invention can be applied to a patient with risk of these diseases, including a patient with hypercholesterolemia.

1. A composition comprising a nucleic acid encoding soluble Flt-1 (sFlt-1) and a pharmaceutically acceptable carrier, wherein said nucleic acid expresses sFlt-1 in an amount effective to inhibit or treat inflammation of vessel wall and/or formation of neointimal hyperplasia.

2. The composition of claim 1, wherein the nucleic acid is inserted in a vector.

3. The composition of claim 2, wherein the vector is selected from the group consisting of a plasmid, an adenovirus vector, and a Hemagglutinating virus of Japan envelope (HVJ-E) vector.

4. The composition of claim 2, wherein the vector is a eukaryotic expression plasmid.

5. The composition of claim 1, wherein the nucleic acid encodes a polypeptide comprising an amino acid sequence of SEQ ID NO: 2.

6. The composition of claim 1, wherein the nucleic acid encodes a polypeptide comprising an amino acid sequence of SEQ ID NO: 2 which has one or more amino acid substitution, deletion, addition, and/or insertion, wherein said polypeptide is functionally equivalent to and has at least 65% identity to a polypeptide comprising amino acid sequence of SEQ ID NO: 2.

7. The composition of claim 1, wherein the amount effective to inhibit inflammation of vessel wall and/or formation of neointimal hyperplasia is between about 0.0001 mg and 100 mg per day per patient.

8. The composition of claim 1, wherein the composition is administered to a patient intramuscularly.

9. The composition of claim 1, wherein the composition is administered to a patient with risk of post coronary intervention restenosis, atherosclerosis, arteriosclerosis, or edema.

10. The composition of claim 9, wherein the patient is a hypercholesterolemia patient.

11. Use of a nucleic acid encoding soluble Flt-1 (sFlt-1) for the production of a pharmaceutical composition for inhibiting or treating inflammation of vessel wall and/or formation of neointimal hyperplasia.

12. The use of claim 11, wherein the nucleic acid is inserted in a vector.

13. The use of claim 12, wherein the vector is selected from the group consisting of a plasmid, an adenovirus vector, and a Hemagglutinating virus of Japan envelope (HVJ-E) vector.

14. The use of claim 12, wherein the vector is a eukaryotic expression plasmid.

15. The use of claim 11, wherein the nucleic acid encodes a polypeptide comprising an amino acid sequence of SEQ ID NO: 2.

16. The use of claim 11, wherein the nucleic acid encodes a polypeptide comprising an amino acid sequence of SEQ ID NO: 2 which has one or more amino acid substitution, deletion, addition, and/or insertion, wherein said polypeptide is functionally equivalent to and has at least 65% identity to a polypeptide comprising amino acid sequence of SEQ ID NO: 2.

17. The use of claim 11, wherein the composition is administered at a dose between about 0.0001 mg and 100 mg per day per patient.

18. The use of claim 11, wherein the composition is administered to a patient intramuscularly.

19. The use of claim 11, wherein the composition is administered to a patient with risk of post coronary intervention restenosis, atherosclerosis, arteriosclerosis, or edema.

20. The use of claim 19, wherein the patient is a hypercholesterolemia patient.

21. A method for inhibiting or treating inflammation of vessel wall and/or formation of neointimal hyperplasia, comprising administration of a nucleic acid encoding soluble Flt-1 (sFlt-1) to a patient in need thereof.

22. The method of claim 21, wherein the nucleic acid is inserted in a vector.

23. The method of claim 22, wherein the vector is selected from the group consisting of a plasmid, an adenovirus vector, and a Hemagglutinating virus of Japan envelope (HVJ-E) vector.

24. The method of claim 22, wherein the vector is a eukaryotic expression plasmid.

25. The method of claim 21, wherein the nucleic acid encodes a polypeptide comprising an amino acid sequence of SEQ ID NO: 2.

26. The method of claim 21, wherein the nucleic acid encodes a polypeptide comprising an amino acid sequence of SEQ ID NO: 2 which has one or more amino acid substitution, deletion, addition, and/or insertion, wherein said polypeptide is functionally equivalent to and has at least 65% identity to a polypeptide comprising amino acid sequence of SEQ ID NO: 2.

27. The method of claim 21, wherein the nucleic acid is administered at a dose between about 0.0001 mg and 100 mg per day per patient.

28. The method of claim 21, wherein the nucleic acid is administered intramuscularly.

29. The method of claim 21, wherein the patient has risk factors for post coronary intervention restenosis, atherosclerosis, arteriosclerosis, or edema.

30. The method of claim 29, wherein the patient is a hypercholesterolemia patient.


SEQUENCE LISTING (8 sequences)

SEQ ID NO: 1 - DNA, 2651 bp, Homo sapiens
  CDS (250)...(2313)
gcggacactcctctcggctcctccccggcagcggcggcggctcggagcgggctccggggctcgggtgcagcggccagcgggcctggcggcgaggattacccggggaagtggttgtctcctggctggagccgcgagacgggcgctcagggcgcggggccggcggcggcgaacgagaggacggactctggcggccgggtcgttggccgggggagcgcgggcaccgggcgagcaggccgcgtcgcgctcaccatggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagaggtgagcactgcaacaaaaaggctgttttctctcggatctccaaatttaaaagcacaaggaatgattgtaccacacaaagtaatgtaaaacattaaaggactcattaaaaagtaacagttgtctcatatcatcttgatttattgtcactgttgctaactttcaggctcggaggagatgctcctcccaaaatgagttcggagatgatagcagtaataatgagacccccgggctccagctctgggccccccattcaggccgagggggctgctccggggggccgacttggtgcacgtttggatttggaggatccctgcactgccttctctgtgtttgttgctcttgctgttttctcctgcctgataaacaacaacttgggatgatcctttccattttgatgccaacctctttttatttttaagcggcgccctatagt

SEQ ID NO: 2 - protein, 687 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRGEHCNKKAVFSRISKFKSTRNDCTTQSNVKH

SEQ ID NO: 3 - DNA, 3394 bp, Mus musculus
  CDS (252)...(2318)
agcgcggaggcggacactcccgggaggtagtgctagtggtggtggctgctgctcggagcgggctccgggactcaagcgcagcggctagcggacgcgggacggcgtggatccccccacaccacccccctcggctgcaggcgcggagaagggctctcgcggcgccaagcagaagcaggaggggaccggctcgagcgtgccgcgtcggcctcggagagcgcgggcaccggccaacaggccgcgtcttgctcaccatggtcagctgctgggacaccgcggtcttgccttacgcgctgctcgggtgtctgcttctcacaggatatggctcagggtcgaagttaaaagtgcctgaactgagtttaaaaggcacccagcatgtcatgcaagcaggccagactctctttctcaagtgcagaggggaggcagcccactcatggtctctgcccacgaccgtgagccaggaggacaaaaggctgagcatcactcccccatcggcctgtgggagggataacaggcaattctgcagcaccttgaccttggacacggcgcaggccaaccacacgggcctctacacctgtagatacctccctacatctacttcgaagaaaaagaaagcggaatcttcaatctacatatttgttagtgatgcagggagtcctttcatagagatgcacactgacatacccaaacttgtgcacatgacggaaggaagacagctcatcatcccctgccgggtgacgtcacccaacgtcacagtcaccctaaaaaagtttccatttgatactcttacccctgatgggcaaagaataacatgggacagtaggagaggctttataatagcaaatgcaacgtacaaagagataggactgctgaactgcgaagccaccgtcaacgggcacctgtaccagacaaactatctgacccatcggcagaccaatacaatcctagatgtccaaatacgcccgccgagcccagtgagactgctccacgggcagactcttgtcctcaactgcaccgccaccacggagctcaatacgagggtgcaaatgagctggaattaccctggtaaagcaactaagagagcatctataaggcagcggattgaccggagccattcccacaacaatgtgttccacagtgttcttaagatcaacaatgtggagagccgagacaaggggctctacacctgtcgcgtgaagagtgggtcctcgttccagtctttcaacacctccgtgcatgtgtatgaaaaaggattcatcagtgtgaaacatcggaagcagccggtgcaggaaaccacagcaggaagacggtcctatcggctgtccatgaaagtgaaggccttcccctccccagaaatcgtatggttaaaagatggctcgcctgcaacattgaagtctgctcgctatttggtacatggctactcattaattatcaaagatgtgacaaccgaggatgcaggggactatacgatcttgctgggcataaagcagtcaaggctatttaaaaacctcactgccactctcattgtaaacgtgaaacctcagatctacgaaaagtccgtgtcctcgcttccaagcccacctctctatccgctgggcagcagacaagtcctcacttgcaccgtgtatggcatccctcggccaacaatcacgtggctctggcacccctgtcaccacaatcactccaaagaaaggtatgacttctgcactgagaatgaagaatcctttatcctggatcccagcagcaacttaggaaacagaattgagagcatctctcagcgcatgacggtcatagaaggaacaaataagacggttagcacattggtggtggctgactctcagacccctggaatctacagctgccgggccttcaataaaatagggactgtggaaagaaacataaaattttacgtcacagatgtgccgaatggctttcacgtttccttggaaaagatgccagccgaaggagaggacctgaaactgtcctgtgtggtcaataaattcctgtacagagacattacctggattctgctacggacagttaacaacagaaccatgcaccatagtatcagcaagcaaaaaatggccaccactcaagattactccatcactctgaaccttgtcatcaagaacgtgtctctagaagactcgggcacctatgcgtgcagagccaggaacatatacacaggggaagacatccttcggaagacagaagttctcgttagaggtgagcactgcggcaaaaaggccattttctctcggatctccaaatttaaaagcaggaggaatgattgtaccacacaaagtcatgtcaaacattaaaggactcatttgaaaagtaacagttgtctcttatcatctcagtttattgttactgttgctaactttcaggcccagaggaaacgctcctcccaaaatgagtttggacatgataacgtaataagaaagcccagtgccctctgcccggggtgcccgctggcccgggggtgctctgtgggccgcccggtgtgtgtttggatttgaagatccctgtactctgtttcttttgtgtgtctgctcttctgtcttctgcttcatagcagcaacctgggacgcatgtttttcttccactctgatgccaacctcttttgatatatatatatatttttaagttgtgaagctgaacaaactgaataatttaagcaaatgctggtttctgccaaagacggacatgaataagttaattttttttccagcacaggatgcgtacagttgaatttggaatctgtgtcgggtgtctacctggttttattttttactatttcattttttgctcttgatttgtaaatagttcctggataacaagttataatgcttatttatttgaaacttggttgttttgttgttttttttttcttttcatgaagtatattgatcttaaactggagggttctaagatgggtcccaggggctcaagatgttgatgtcattccgagagtaaagctatgtcccaatgtgaattatgaaggtccagcaggtctgctccaccctcctctgtccacccaggtaattacacgtgtgtttcctgctgtgttagatgctgttcctcattgtccttggctggactgacagcccctgactgacggcaaaagtgcagcaagccttcattataaacactcatggcccctgggcactgttttaaagcccttcaccaagctttgatggcattcaaagatgtccacaacccatgtatccaggatataaaggctattgtgagtggagatttaatgcaatcttcttaatgtctattgaaaaatctacccatgagagaaagaaaagtccaccttctctatatgcaaatgttttatggggattaagaaattgcaaaagctaagaaattacaaaaaaaaaaaaaaaaa

SEQ ID NO: 4 - protein, 688 aa, Mus musculus
MVSCWDTAVLPYALLGCLLLTGYGSGSKLKVPELSLKGTQHVMQAGQTLFLKCRGEAAHSWSLPTTVSQEDKRLSITPPSACGRDNRQFCSTLTLDTAQANHTGLYTCRYLPTSTSKKKKAESSIYIFVSDAGSPFIEMHTDIPKLVHMTEGRQLIIPCRVTSPNVTVTLKKFPFDTLTPDGQRITWDSRRGFIIANATYKEIGLLNCEATVNGHLYQTNYLTHRQTNTILDVQIRPPSPVRLLHGQTLVLNCTATTELNTRVQMSWNYPGKATKRASIRQRIDRSHSHNNVFHSVLKINNVESRDKGLYTCRVKSGSSFQSFNTSVHVYEKGFISVKHRKQPVQETTAGRRSYRLSMKVKAFPSPEIVWLKDGSPATLKSARYLVHGYSLIIKDVTTEDAGDYTILLGIKQSRLFKNLTATLIVNVKPQIYEKSVSSLPSPPLYPLGSRQVLTCTVYGIPRPTITWLWHPCHHNHSKERYDFCTENEESFILDPSSNLGNRIESISQRMTVIEGTNKTVSTLVVADSQTPGIYSCRAFNKIGTVERNIKFYVTDVPNGFHVSLEKMPAEGEDLKLSCVVNKFLYRDITWILLRTVNNRTMHHSISKQKMATTQDYSITLNLVIKNVSLEDSGTYACRARNIYTGEDILRKTEVLVRGEHCGKKAIFSRISKFKSRRNDCTTQSHVKH

SEQ ID NO: 5 - DNA, 20 bp, Artificial Sequence
  An artificially synthesized primer sequence.
ggatccatgaactttctgct

SEQ ID NO: 6 - DNA, 22 bp, Artificial Sequence
  An artificially synthesized primer sequence.
gaattcaccgcctcggcttgtc

SEQ ID NO: 7 - DNA, 18 bp, Artificial Sequence
  An artificially synthesized primer sequence.
atggatgacgatatcgct

SEQ ID NO: 8 - DNA, 19 bp, Artificial Sequence
  An artificially synthesized primer sequence.
atgaggtagtctgctaggt